(19) United States
(12) Patent Application Publication
(10) Pub. No.: US 2011/0160625 A1
(43) Pub. Date: Jun. 30, 2011

(54) Apparatus and method for training knee and hip joints of a patient in post-traumatic or post-operative period

(76) Inventor: Nikolay Yefimov, San Francisco, CA (US)

(21) Appl. No.: 12/655,257

(22) Filed: Dec. 28, 2009

(51) Int. Cl. A61H 1/02; A61F 5/00

(52) U.S. Cl. 601/5; 602/26

(57) ABSTRACT

The apparatus for training knee and hip joints of a patient in the post-traumatic or post-operative period makes it possible to perform controllable bending and stretching motions of the leg or with use of a knee-immobilization device. The patient's foot of the injured leg is secured on a foot pedal, and the motions of the foot are used as a motive force to drive a carriage that supports the foot pedal. Ankle pump motions of the foot pedal are transmitted by means of a ratchet mechanism to a drum that is used for winding a cord. One end of the cord is attached to the drum, and another end can be selectively attached either to the rear crossbar or to the knee-immobilizing device. Depending on the position of the free end of the cord, the disconnectable pulley and direction of motion of the carriage, the injured leg can be stretched or bent.

### FIELD OF THE INVENTION

The present invention relates to an apparatus and method for rehabilitative exercise of the leg and, more particularly, to an apparatus and method for converting the motions of a healthy foot of an injured leg into positively driven motions such as controlled bending or stretching of the leg with articulation through the knee or hip joint. The pivotal motions of the foot are converted into linear motions of a leg-supporting carriage or into angular movement of a thigh though a conversion mechanism consisting of a clutch, ratchet wheel, pulling cord, etc.

### BACKGROUND OF THE INVENTION

Not accounting for knee or hip joints, in the United States, alone, there are over one million anterior crucial ligament knee surgeries and knee replacement surgeries every year. Outside the U.S., however, over ten million such surgeries are performed annually. Immediate post-operative rehabilitation of the knee or hip joint is desirable following many surgical procedures, including total joint replacement or joint reconstruction, to restore the joint to its full range of motion. Effective rehabilitation requires controlled movement of the knee or hip joint as soon as possible after surgery without bearing weight on the joint or placing a substantial force load on the joint. As rehabilitation progresses, the range of joint motions can be increased, and force loads can be applied to the joints.

Many apparatuses that are aimed at meeting the above objectives are known in the art. For example, U.S. Pat. No. 4,844,454 issued in 1989 to S. Rogers discloses a self-operable knee therapy device that includes a first elongate platform on which the lower leg of a person is secured so that the back of the leg contacts the platform, a second elongate platform pivotally joined at one end to an end of the first platform and on which the upper leg of the person is secured with the back of the upper leg being in contact with the second platform, and an elongate central support member pivotally joined at its upper end to the joined ends of the first and second platforms for supporting the joined ends in an elevated position. A handle is coupled to the first platform to enable grasping by a user so that when the handle is moved away from the person, the free end of the first platform is caused to pivot downwardly, and when the handle is moved toward the person, the free end is caused to pivot upwardly. Manual movement of the handle causes the lower leg to be pivoted relative to the upper leg in a selective manner to exercise and rehabilitate the knee and/or leg muscles.

U.S. Pat. No. 4,492,222 issued in 1985 to M. Hajianpour discloses a knee exerciser that cyclically flexes a patient's knee by alternately extending and retracting a leg support having one end disposed behind the knee. The leg support is hinged at one end to a thigh support and is fixed at its other end to a motor assembly. The motor assembly pivots relative to a frame. The other end of the thigh support is also pivotally secured to the frame. The leg support includes a tubular member secured to the thigh support hinge and a screw, which is rotatably driven by the motor and is engaged in the tube by means of threading. Rotation of the screw extends and retracts the leg support, thereby moving the leg/thigh hinge to cyclically flex the knee joint. The motor can be controlled to set the limit point of the flexure cycle and to control the speed of the motor.

A device for rehabilitative leg exercise is disclosed also in U.S. Pat. No. 4,974,830 issued to C. Genovese, et al, in 1990. The leg is mounted on a rigid support structure while the knee is driven through alternating flexion and extension by applying an external force to the knee across a mechanical pivot point. Continuous passive-motion devices known in the art, however, require careful anatomical alignment of the rigid support structure with the leg and strict monitoring of external force loads applied to leg joints to prevent post-operative injury to the joint during rehabilitation.

U.S. Pat. No. 5,303,716 issued in 1994 to B. Mason, et al, discloses a portable rehabilitation device employing a suspension system to enable passive or active range of motion exercise of the knee or hip joint. The device base is adapted to support the suspension system while the patient reclines to perform the respective leg exercises. The suspension system includes a linear track that retains three shuttles slidably positioned in series along the track attached to flexion, extension, and leg support lines, respectively. For passive suspension exercise, the leg support line is secured, and the leg is suspended by only a single cuff at the ankle with the knee joint in a position of full extension. For passive motion exercise, both cuffs suspend the leg, and the patient alternately pulls on handles attached to the flexion and extension lines, causing alternate passive flexion and extension of the knee and hip joints. The device can be driven by leg or by leg and hand simultaneously.

U.S. Pat. No. 5,687,742 issued in 1997 to L. Johnson discloses a knee extension device and method for rehabilitating an injured knee by gradually extending the knee into a straightened position. The device includes an elongated member, a lower leg support attached to the elongated member, and a pressure-applying strap capable of being wrapped around the elongated member. The leg is positioned on the elongated member, with the lower portion of the leg resting on the support member. Pressure is selectively applied to the leg using the pressure-applying strap to gradually force the knee toward a straightened position.

### SUMMARY OF THE INVENTION

With regard to the device of the invention, bending and stretching of the knee joint can be carried out with use of a knee-immobilization device. The patient's foot is secured to a foot pedal and drives the sliding carriage along the frame in the forward or reverse direction (i.e., leg-stretching or bending direction) by using motions of the healthy foot around the ankle joint. The force of the foot is transmitted by means of the ratchet wheel to the drum, on which one end of the cord is secured and onto which the cord is wound. The other end of the cord is guided by a pulley, which is attached either to a stationary front crossbar or to the carriage. Rotation is transmitted from the foot pedal to the ratchet wheel by means of a lever, and direction of the ratchet wheel rotation depends on the position of pawls in the ratchet pawl mechanism. This mechanism has two ratchet pawls that can be switched in an alternating manner by a toggle mechanism between the first position, in which the first pawl engages one side of the ratchet wheel for rotation in the clockwise direction, and the second position, in which the second pawl engages the ratchet wheel on the other side for rotation of the ratchet wheel in the counterclockwise direction. On the side facing the cord drum, the ratchet wheel has a first half clutch, while the second half clutch is formed on the facing side of the cord drum. The drum is slidingly supported on the ratchet wheel shaft and can be shifted in the axial direction of the ratchet shaft toward the ratchet to engage the half clutches for transmitting rotation of the ratchet to the cord drum. Axial displacement of the drum in the clutch engagement direction is carried out by means of a flexible steel cable, the control of which is held in the patient's hands.

Thus, by attaching the front pulley either to the carriage or to the front crossbar and by attaching the free end of the cord either to the rear crossbar of stationary frame or to a knee brace on the patient's leg, it becomes possible to move the sliding carriage with the foot secured on the foot pedal in the leg-stretching or leg-bending direction.

Knee bending and stretching exercises performed with the use of the apparatus of the invention provide the following effects:
1. Maintain muscle tone
2. Restore mobility in knee and hip joints
3. Improve blood circulation through tissues and leg muscles
4. Prevent formation of thromboses in blood vessels of injured leg
5. Reduce edema in knee and hip joints
6. Prevent “freezing” of joints in post-operative period
7. Shorten time required for healing and restoration of normal functioning of knee and hip joints

### BRIEF DESCRIPTION OF DRAWINGS

FIG. 1 is three-dimensional view of the apparatus in the unfolded state and in the ready-for-use position.

FIG. 2 is a longitudinal sectional view of the support-leg extension mechanism.

FIG. 3 is a perspective view of the clutch-and-drum assembly.

FIG. 4 is a longitudinal sectional view of the clutch-and-drum assembly shown in FIG. 3.

FIG. 5 is a side view of the ratchet wheel mechanism.

FIG. 6 is a three-dimensional view of the drum shifting mechanism.

FIG. 7 is a three-dimensional view of the mechanism for attachment of the pulley to the front crossbar or to the carriage.

FIG. 8 shows an example of an angle-controlling knee brace suitable for use in conjunction with the apparatus of the invention during the initial stages of healing.

FIG. 9 shows a simplified knee brace for use at the stage of healing when the injured knee has greater freedom of motion than in the initial stage of healing.

FIG. 10 is a schematic view that illustrates use of the apparatus for a reclined patient.

FIG. 11 is a view of the mechanism for attaching the end of the cord to the frame of the apparatus of the invention.

FIG. 12 is a schematic view of the apparatus in use for a patient in the sitting position, with the frame inclined downward.

FIG. 13 is a schematic view of the apparatus in use for a patient in a sitting position, with the frame in the horizontal position.

FIG. 14 is a schematic view of the apparatus in use for a reclined patient, with the frame inclined upward at a moderate angle of inclination.

FIG. 15 is a schematic view of the apparatus in use for a reclined patient, with the frame inclined upward at a high angle of inclination.

### DETAILED DESCRIPTION OF THE INVENTION

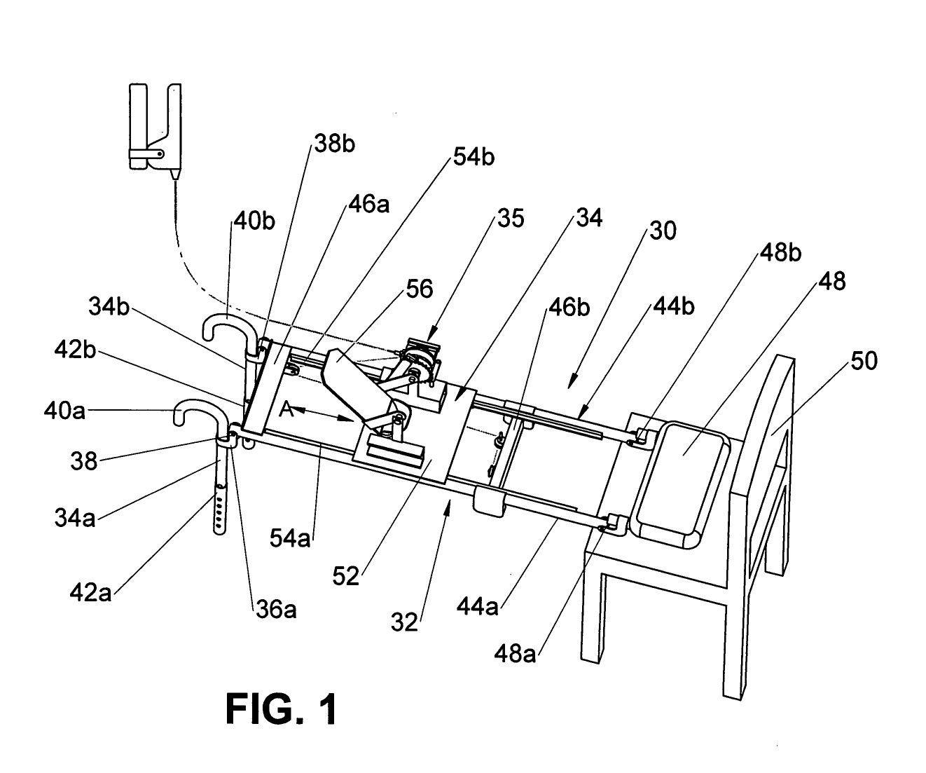

The apparatus of the invention in its entirety is shown in FIG. 1, which is a three-dimensional view of the apparatus in the unfolded state and ready for use. The apparatus as a whole is designated by reference numeral 30 and comprises a frame 32, a carriage 34 moveably installed on the frame, and a carriage drive mechanism 35 installed on the carriage 34.

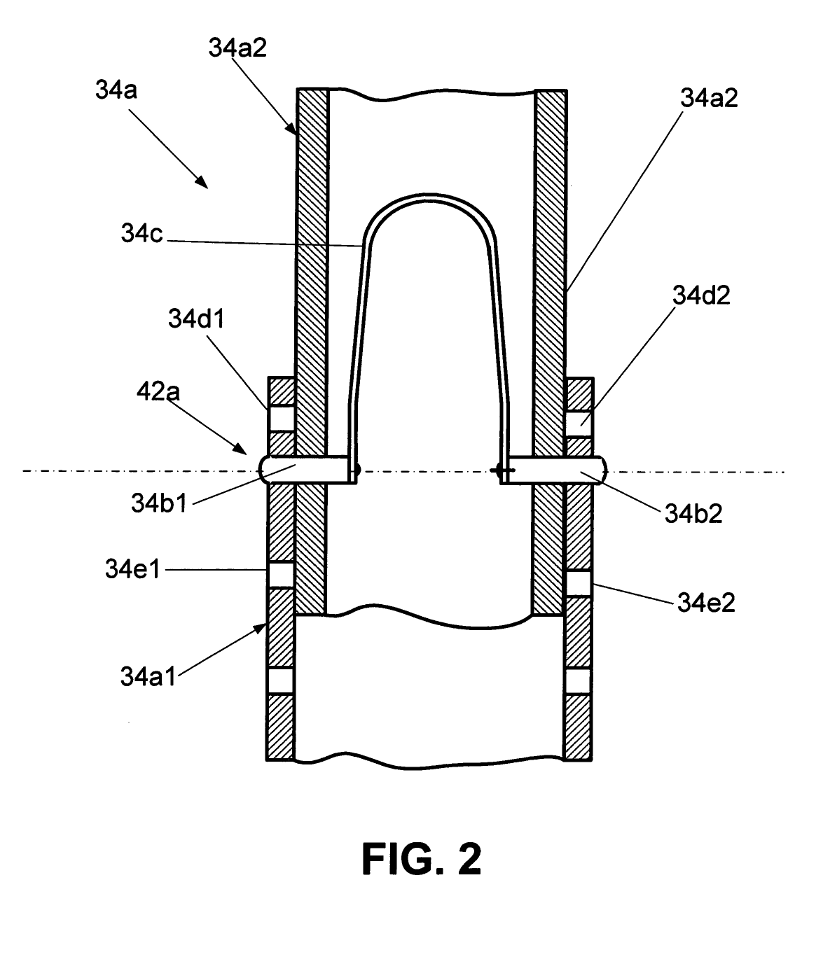

The frame 32 comprises support legs 34a and 34b pivotally attached to the frame 32, e.g., by means of L-shaped links 36a and 36b with rotation on pivots, such as a pivot 38. For attachment to various stationary objects, such as a wall bar or the like, hooks 40a and 40b are provided on the ends of the support legs 34a and 34b, respectively. The support legs are equipped with mechanisms 42a and 42b (FIG. 1) for adjustment of their length by using two telescopically engaged tubular members with spring-loaded lock pins. An example of such a mechanism is shown in FIG. 2, where reference numerals 34a1 and 34a2 designate telescopic tubular members of the support leg 34a. The mechanism contains lock pins 34b1 and 34b2, which are spread apart by a leaf spring 34c. For adjusting the support leg, the pins 34b1 and 34b2 are squeezed toward each other into the inner tube 34a2, and the outer tube 34a1 is shifted into the leg shortening or lengthening direction, and then the lock pins are released and snap into another set of lock openings 34d1, 34d2 or 34c1, and 34c2.

The frame 32 further comprises a pair of longitudinal bars 44a and 44b and a pair of crossbars 46a and 46b. On the side of the crossbar 46b, the frame has a seat 48 for the patient. The seat 48 is pivotally attached to the ends of the longitudinal bars 44a and 44b and can be folded or unfolded by turning the seat on the pivots, such as pivots 48a and 48b (FIG. 1).

In FIG. 1, the frame 32 of the apparatus 30 is shown in the unfolded state, with one end of the frame 32 supported by support legs 34a and 34b that rest on the floor, while the other end of the frame 32 rests on the seat of a chair 50.

The carriage 34 comprises a base plate 52 that is guided along the frame 32 in a direct or reverse direction, as shown by arrow A in FIG. 1. The guides 54a and 54b are attached to the longitudinal bars 44a and 44b of the frame 32, respectively.

The carriage drive mechanism 35 (FIG. 1) is intended to operate from a force developed by the healthy foot of the injured leg of a patient. The mechanism comprises a foot support, e.g., a pedal 56 for the patient's foot on which the foot is secured by straps that are wrapped around the upper side of the foot, overlapped with each other, and secured together, e.g., by “hook-and-loop” fastener of the type available, e.g., from Velcro, USA.

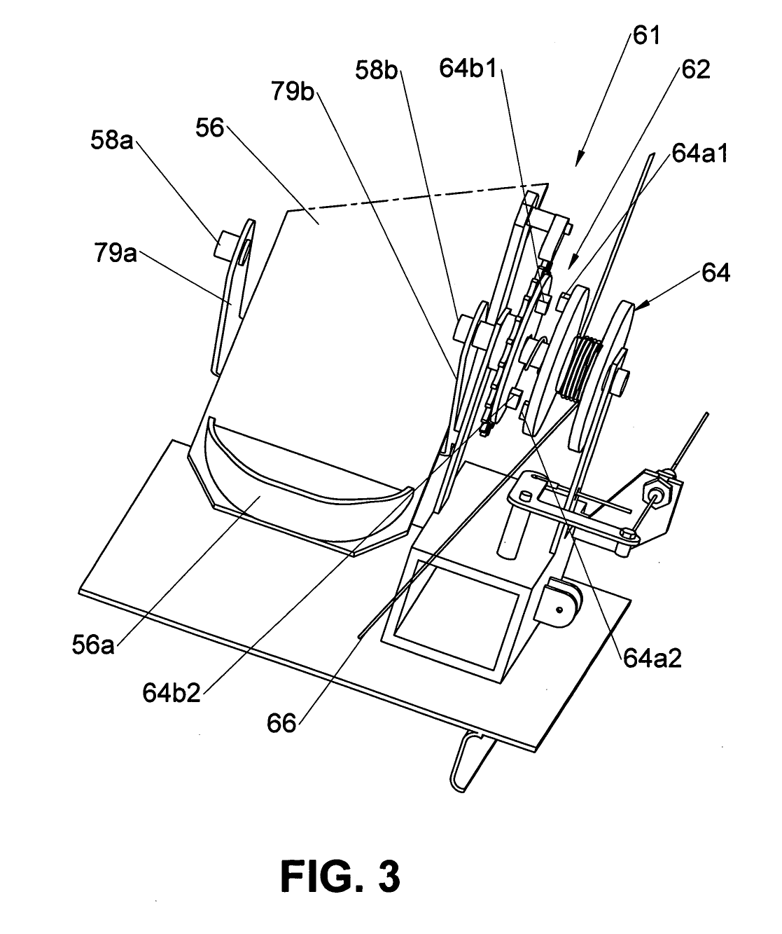

The pedal 56 is pivotally supported in a pendulum manner on pivots 58a and 58b held by vertical stands 79a and 79b, which are seen in FIG. 3. This drawing is a perspective view of a clutch-and-drum assembly used in the apparatus of the invention and described below. The heel of the foot rests on the raised portion 56a of the pedal.

Pivotal motions of the pedal 56 are transmitted to the carriage 34 through a motion conversion mechanism 61 (FIG. 3) that connects the foot support with the carriage for converting angle pump motions of the foot into linear motions of the carriage 34 in the guides 54a and 54b of the frame 32 (FIG. 1).

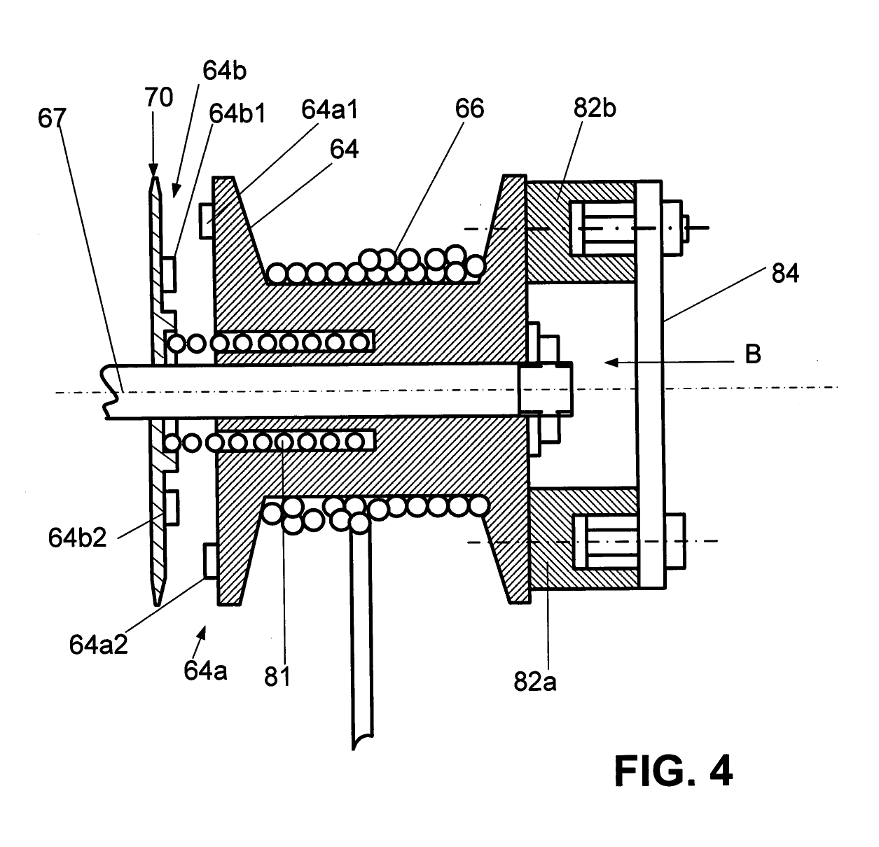

The pivots 58a and 58b are coaxial with the ankle joint and with the longitudinal axis of a clutch-and-drum assembly 62, which is part of the aforementioned motion conversion mechanism 61 and is also supported by the carriage 34. The clutch-and-drum assembly 62 is described in more detail below with reference to FIGS. 3 and 4, where FIG. 4 is a longitudinal sectional view of the assembly.

As shown in FIGS. 3 and 4, the clutch-and-drum assembly 62 contains a drum 64 onto which a cord 66 is wound. One end of the cord is secured to the drum 64, and the free end of the cord extends from the drum and, as described below, is guided through a cord-guiding-and-attachment device that contains a disconnectable pulley which can be attached either to the carriage 34 or to the front crossbar 46a located on one side of the carriage 34 (FIG. 1). The free end of the cord is then secured either to the rear crossbar 46b or to the knee brace (described later) on the patient's leg. The drum 64 is supported by a shaft 67 with possibility of free rotation relative to the shaft 67. One side of the drum forms a first half-clutch 64a (FIG. 4), which faces a second half clutch 64b that is formed on the facing side of a sprocket 70. The sprocket is also supported by the shaft 67 with possibility of free rotation on this shaft.

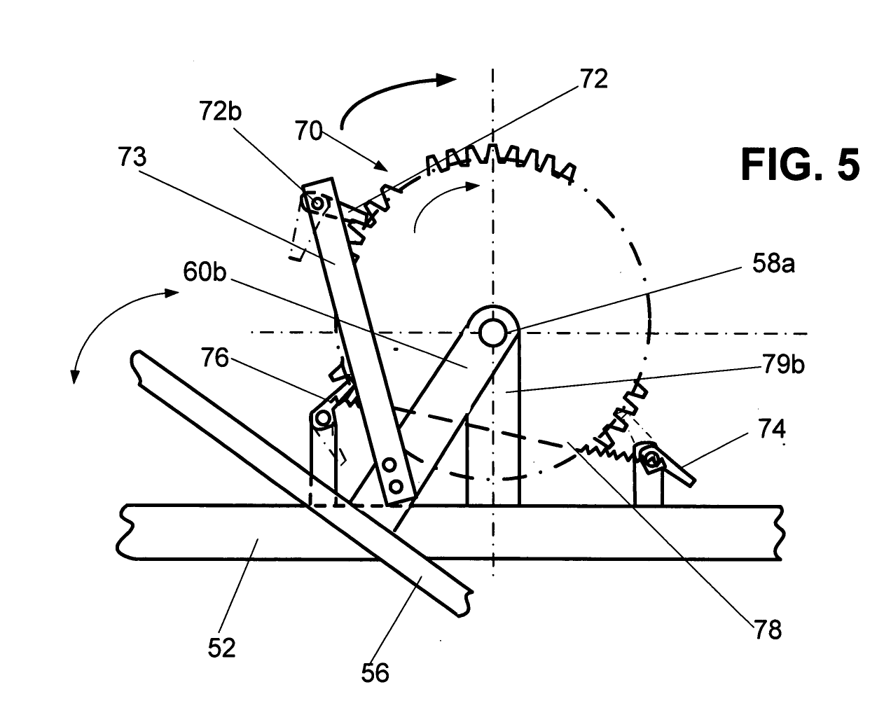

As shown in FIG. 5, which is a side view of the sprocket 70 and the sprocket-support-and-rotation-control mechanism, the sprocket engages pawls 72 and 74 or 72 and 76. The drive pawl 72 is rotationally installed on a pivot 72b (FIG. 5). The pivot 72b is secured to a link 73 (FIG. 5) that is rigidly connected to a link stand 60b that is rigidly connected to the pedal 56. The pedal-supporting link 60b, in turn, is pivotally supported on the carriage platform 52. Thus, when the pawl 72 is in the position shown in FIG. 5, the ankle pump motions of the patient's foot (i.e., swinging motions of the foot up and down about the ankle joint) in the clockwise direction, as shown in FIGS. 1 and 5, rotates the sprocket 70 in the clockwise direction. Rotation in the counterclockwise direction is prevented by the lock pawl 76, the locking pawl 74 being in inoperative position, as shown by solid lines. When the pawl 72 is switched to the position shown by the dot-and-dash lines in FIG. 5, rotation of the pedal in the counterclockwise direction rotates the sprocket 70 in the counterclockwise direction. Rotation in the clockwise direction is prevented by the pawl 74, shown by the dot-and-dash lines in FIG. 5, the pawl 76 being turned into the inoperative position, also shown by the dot-and-dash lines in FIG. 5. This is achieved by connecting the pawls with an extension spring 78 that switches one of the lock pawls 74 or 76 into the inoperative position when the other of these pawls is manually switched to the operative position.

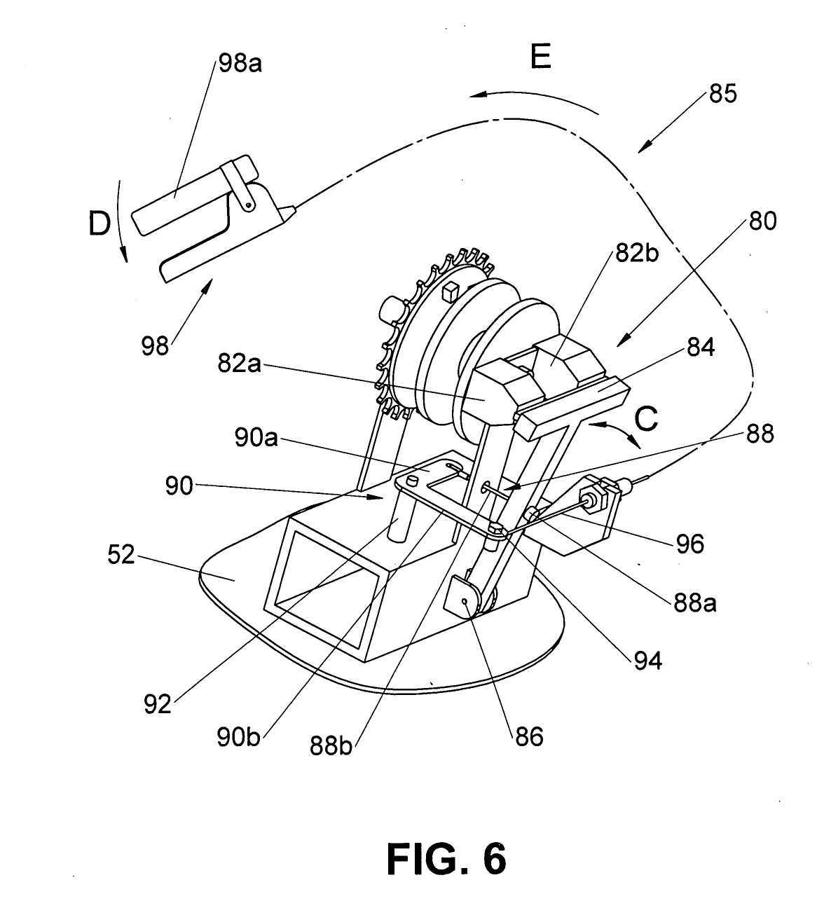

A clutch-switching engagement/disengagement mechanism 80 will now be described with reference to FIGS. 4 and 6, where FIG. 6 is a three-dimensional view of a drum shifting mechanism 85. As described above in the embodiment of the apparatus of the invention shown in FIGS. 1 to 5, the clutch of the clutch-and-drum assembly 62 consists of the first half-clutch 64a, which is formed on the end face of the drum 64, and the second half-clutch is formed on the sprocket 70. The drum 64 and the sprocket can freely rotate on the shaft 67, and the drum 64 slides on the shaft 67 back and forth in the axial direction, shown in FIG. 4 by arrow B.

In the disengaged state of the clutch, the drum is shifted to its rearmost position by the spring 81 (FIG. 4), and the end-face of the drum 64 that is opposite to the first half-clutch rests on a pair of pushing shoes 82a and 82b, which are attached to one end of a lever 84 that belongs to the drum-shifting mechanism 85 (FIG. 6). The opposite end of the lever 84 is pivotally supported on the base plate 52 of the carriage 34 (FIGS. 1 and 6) and can swing in the direction of arrow C (FIG. 6) on a pivot 86. A pull link 88 passes through the lever 84 so that a head 88a of the pull link rests on the outer surface of the lever 84 while the elongated part 88b of the pull link projects in the direction of the sprocket (FIG. 6) and parallel to the shaft 67 (FIG. 4). The free end of the elongated part 88b of the pull link is pivotally connected to one arm 90a of an L-shaped lever 90 that is rotationally installed on an axle 92 that is secure to the base plate 52 of the carriage 34. The other arm 90b of the L-shaped lever is pivotally connected by a pivot 94 to an end of a flexible metal cable 96 of the type used for bicycle brake control. The opposite end of the flexible metal cable 96 is connected to a handle 98 that is held in the hand of the patient (not shown).

Thus, when it is necessary to engage the half-clutches and thus kinematically connect the drum 64 with the sprocket 70 for joint rotation with the latter, the patient pushes a control lever 98a of the handle 98 in the direction of arrow D (FIG. 6), the flexible metal cable 96 moves rearward in the direction of arrow E, the L-shaped lever 90 turns in the counterclockwise direction around the pivot 92 (FIG. 6), the head 88a of the pull link 88 pulls lever 84, the latter turns in the counterclockwise direction shown by arrow C in FIG. 6 around the pivot 86, and the pushing shoes 82a and 82b push on the rear end face of the drum 64 (FIG. 4) against the force of the spring 81 and shift the drum forward toward the sprocket 70. If the sprocket 70 rotates with drive from the pedal 56 through the carriage drive mechanism 35 and the ratchet mechanism shown in FIG. 5, the projections 64a1 and 64a2 (FIG. 4) of the first half-clutch 64a engage the projections 64b1 and 64b2 of the second half-clutch 64b, and the latter transmits rotation of the sprocket to the drum 64. Depending on the direction of rotation of the drum, which, in turn, depends on the position of the pawls 72b, 74, and 76 in the sprocket mechanism shown in FIG. 5, the cord 66 is wound on the drum 64 in one or the other direction.

Depending on the direction of movement of the carriage 34 relative to the patient (not shown), the free end of the cord 66 is guided over a pulley that is attached either to the front crossbar 46a shown in FIG. 1 (during leg stretching) or to the carriage (during leg bending). After guiding through the pulley, the end of the cord is attached either to the rear crossbar 46b (during leg bending) or to the knee brace (during knee stretching).

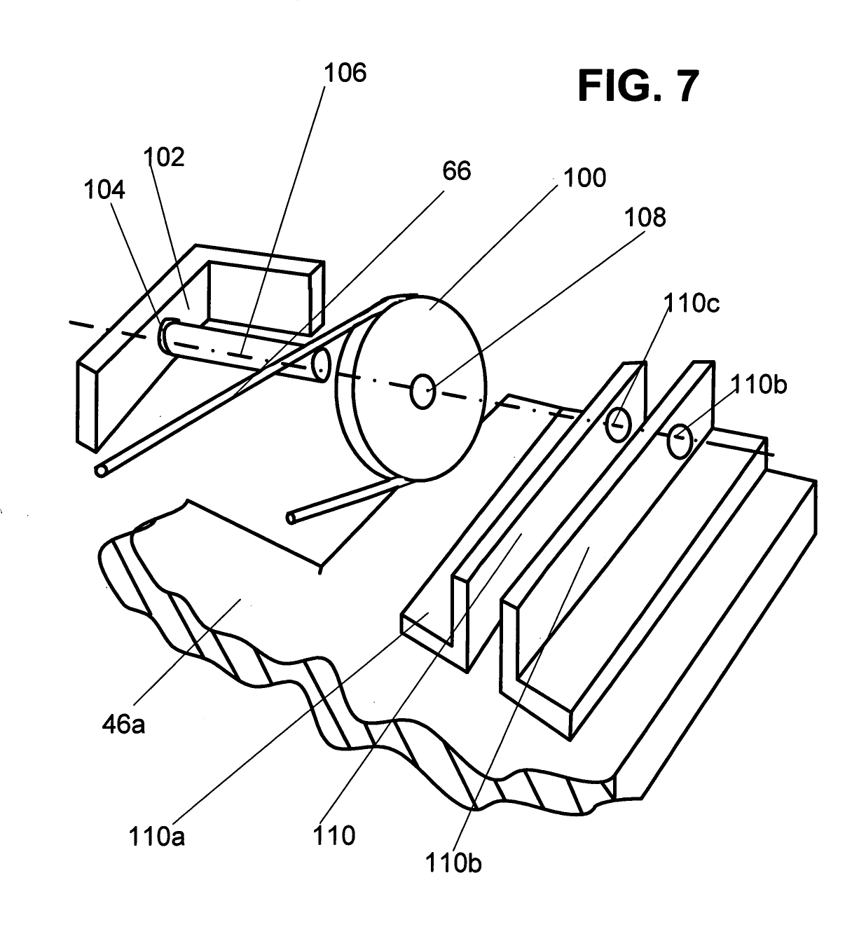

Positions and points of attachment of the cord 66 on the apparatus of the invention or on the patient's leg are explained later in connection with the operation of the apparatus and methods of treatment. Shown in FIG. 7 is an example of a cord-guiding-and-attachment device for attachment of a cord-guiding pulley to the front crossbar or to the carriage. The device consists of a pulley 100 for guiding the cable 66 and an L-shaped bracket 102 with an opening 104 for insertion of an axle 106 that further passes through the central opening 108 of the pulley 100. The pulley is inserted into a slot 110 formed between two brackets 110a and 110b secured, e.g., the crossbar 46a (FIGS. 1 and 7) of the frame 32 or to the carriage 34. The brackets 110a and 110b have openings 110c and 110d, respectively. When the opening 108 of the pulley 100 is arranged coaxially to the opening 110c and 110d, the axle 106 passes through all of these openings and secures the pulley 100 in the arms 110a and 110b on the crossbar of the frame of on the carriage. The L-shaped bracket 102 is intended to protect the cord from disconnection from the pulley.

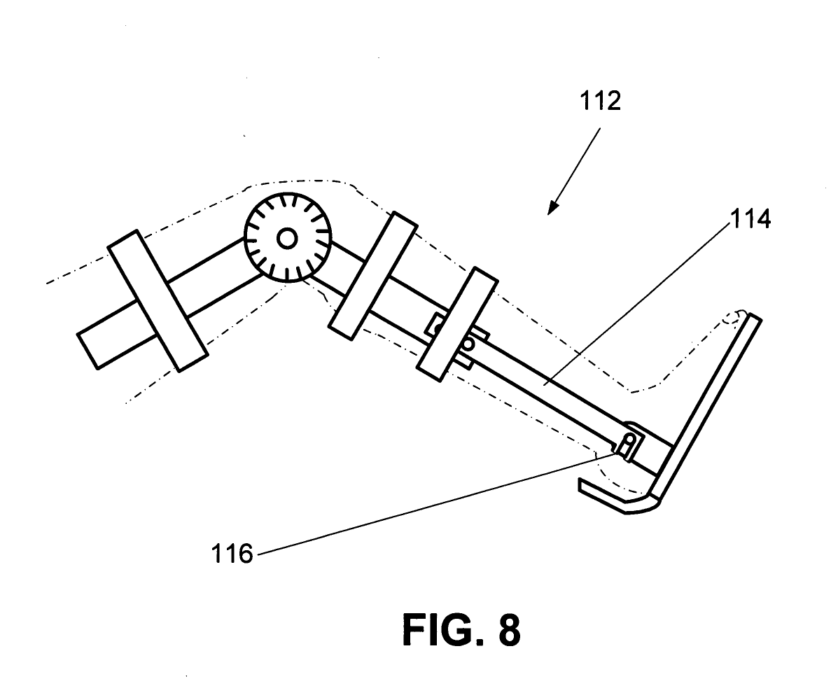

Other auxiliary devices needed for operation and efficient use of the apparatus of the invention are knee-restricting orthopedic devices such as splints or knee immobilizers of the type described in U.S. Pat. No. 7,235,059 issued in 2007 to J. Mason, et al. The device 112 comprises an orthopedic brace having an adjustable range of rotation in the extension and flexion directions, as shown in FIG. 8. The orthopedic brace of the aforementioned type is used to reduce the load on the knee and to restrict lateral motions of the knee during the initial period directly after injury or operation. According to the present invention, the device of U.S. Pat. No. 7,235,059 is modified by attachment of extension bars 114 (only one of which is seen in FIG. 8). Such a device is fixed on the patient's leg and is used to secure the damaged or post-operative knee joint in the anatomically correct position. By means of hooks 116 on the ends of the extension bars 114, the device is secured to the carriage 34 of the apparatus. In this case, during the operation the longitudinal force transferred from the angle joint to the hip joint by-assing the knee joint.

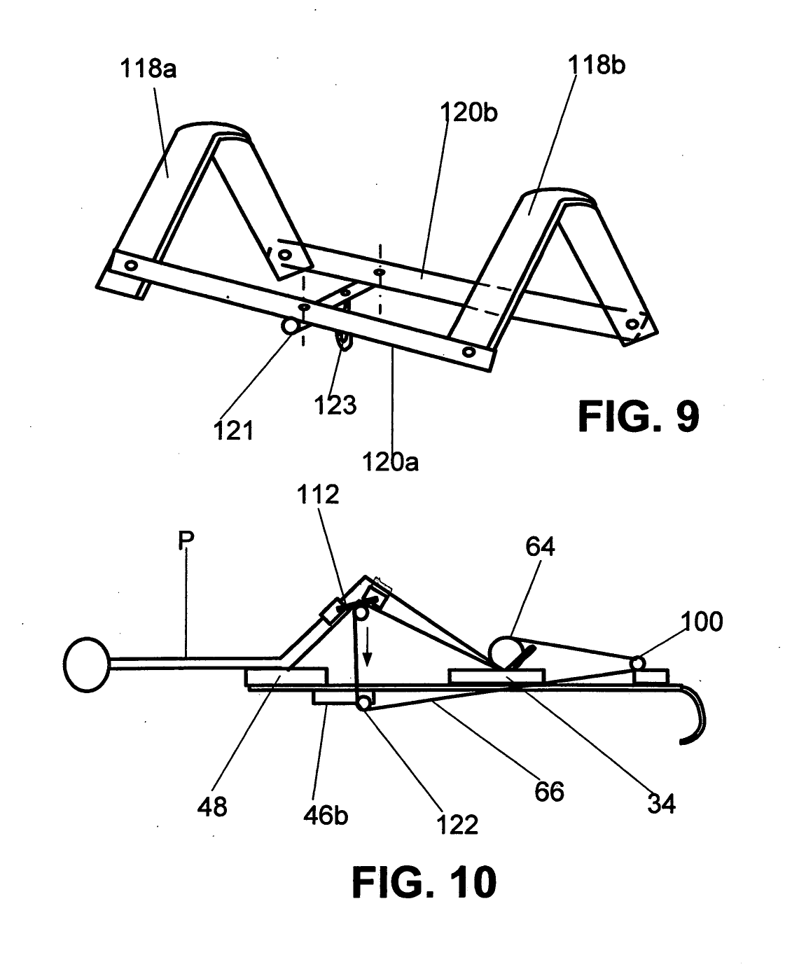

FIG. 9 shows a simplified knee brace that is intended for use rather than the knee brace shown in FIG. 8 at later stages of recovery when the knee joint has a greater degree of freedom. The device, which is designated by reference numeral 118, consists of two straps 118a and 188b connected by rod bars 120a and 120b. The rod bars 120a and 120b are also connected by a crossbar 121. In use, the straps are placed onto the upper side of the leg so that the straps are arranged above and below the knee joint, and then the crossbar 121 is connected to the rod bars 120a and 120b on the lower side of the leg. The crossbar 121 has a hook 123 to which the free end of the cord 66 is to be attached.

The following describes operation of the device and its use at various stages of the patient's healing process.

The procedure shown in FIG. 10 is recommended during the early stage of recovery when movement of the knee must be strictly controlled and limited. More specifically, the knee is placed into the orthopedic brace 112 of the type shown in FIG. 8. In the position shown in FIG. 10, the patient P performs exercises lying on the back. For the stretching operation, the pulley 100 (FIGS. 7 and 10) is attached to the front crossbar 46a (FIG. 1). The cord 66 is guided from the drum 64 (FIG. 4) through a pulley 100, which is attached to the front crossbar 46a, and the other end of the cord is attached to the knee brace 112 (during leg stretching).

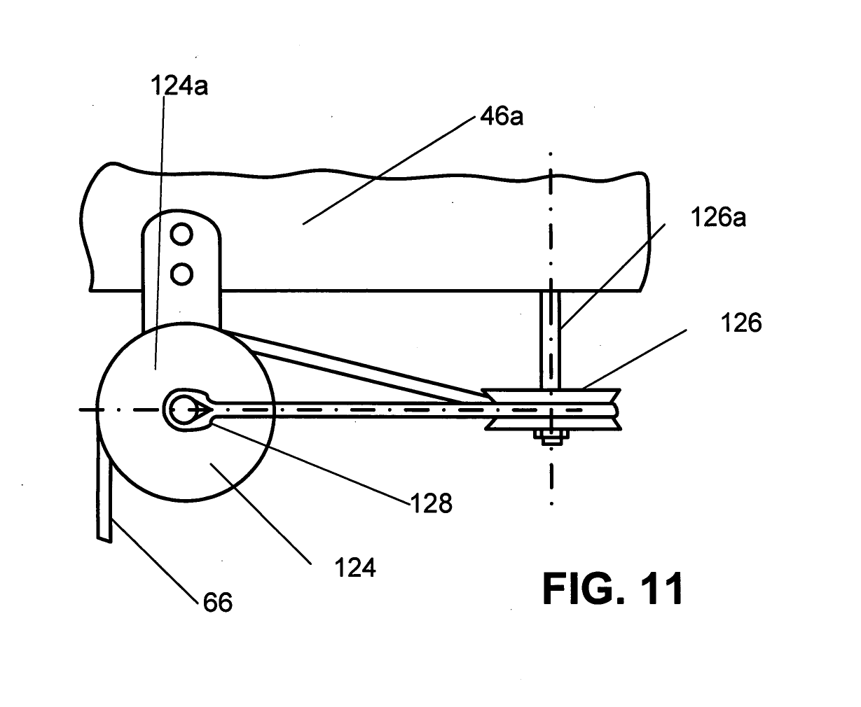

An example of the device that connects the cord 66 to the rear crossbar 46b is shown in FIG. 11. The device can be easily disconnected and consists of two pulleys 124 and 126 installed on mutually perpendicular axles 124a and 126a, which are fixed to the crossbar 46b. The end of the cable is made in the form of a loop 128 and during leg bending is secured to the end of the axle 124a.

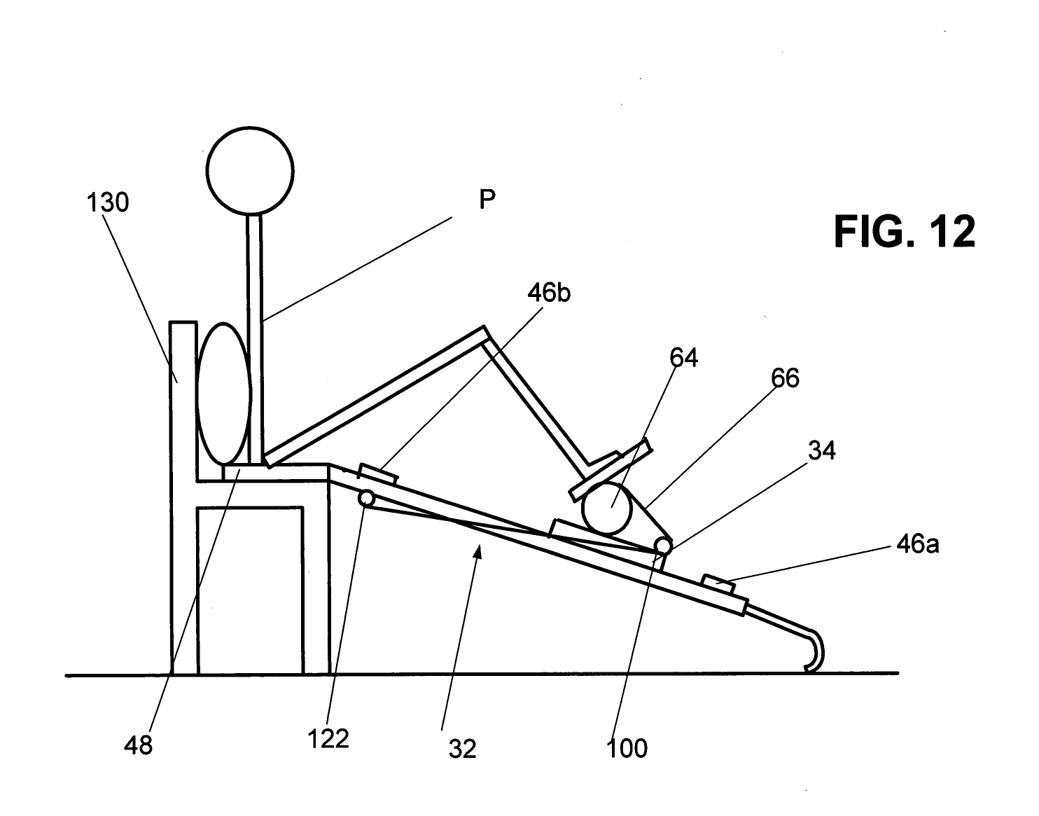

FIG. 12 shows the exercise to be done in the sitting position. In this case, the seat 48 of the apparatus is placed on the seat of the chair 130, and the opposite end of the frame 32 rests on the floor. The cord 66 is guided from the drum 64 (FIG. 4) through a pulley 100, which is attached to the carriage 34 to a pulley 122, and then the end of the cord is attached to the rear crossbar 46b (during leg bending) or to the knee brace (during leg stretching)with the pulley 100 being attached to the front crossbar (not shown in FIG. 12).

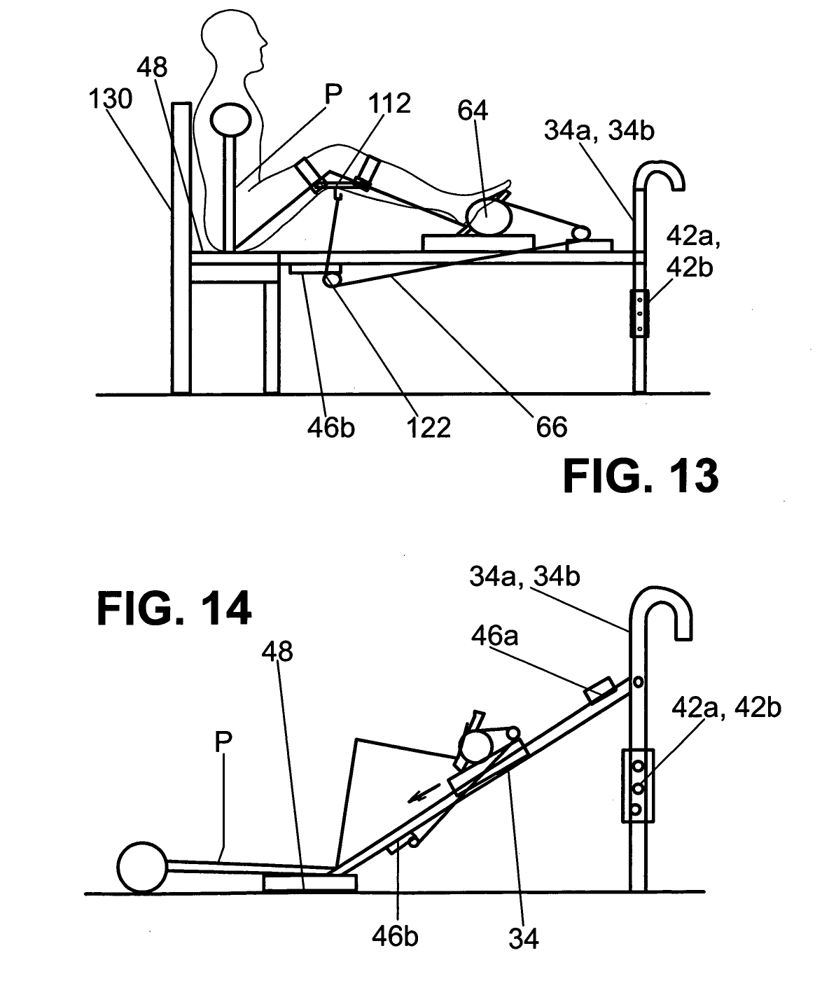

FIG. 13 shows the frame arranged in the horizontal position. The patient P sits on the chair 130. FIG. 13 shows the leg-stretching exercise for which the end of the cord 66 is attached to the hook 123 (FIG. 9) of the knee brace 118, and the pulley 100 is attached to the front crossbar 46a.

FIG. 14 shows the situation, which is the same as that shown in FIG. 10 but with the frame inclined upward from the seat 48 of the apparatus at a moderate angle. The patient P lies on his/her back. In this case, the angle a of inclination of the frame 32 is adjusted by means of the support-leg adjustment mechanisms 42a and 42b shown in FIGS. 1 and 2. The exercises shows leg bending.

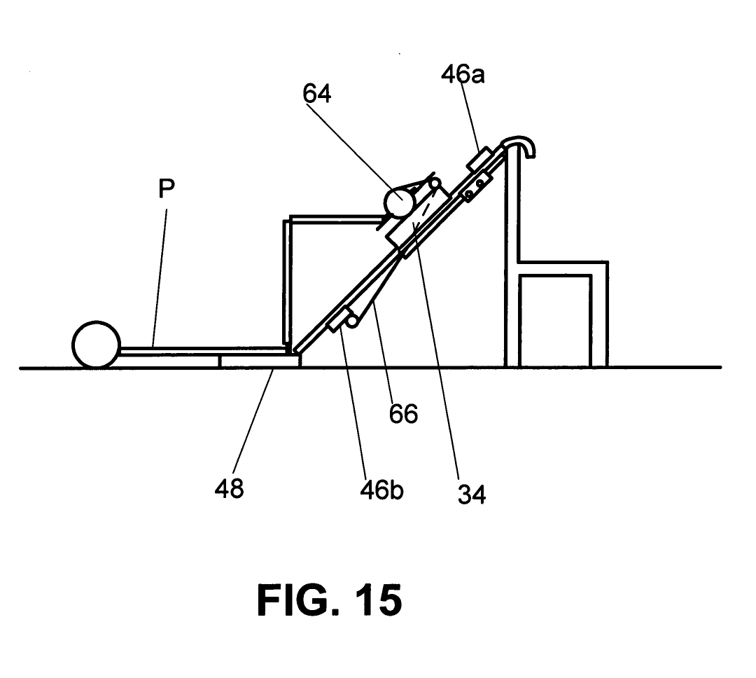

FIG. 15 shows the situation to be used during the final stage of healing. The patient P lies on his/her back, and the frame 34 of the apparatus is inclined upward at a high angle with support of the frame end on the upper edge of the chair back. The exercise is intended mainly for stretching muscles and ligaments on the back side of the leg.

Depending on the positions of the pawl 72 and pawls 74 and 76 (FIG. 5), ankle pump movement of the foot allows plantar flexion and dorsiflexion. Plantar flexion is the movement that increases the 90-degree angle between the front part of the foot and the shin, as done when depressing an automobile pedal. The word “plantar” is commonly understood in medical terminology as the bottom of the foot and is translated as “toward the sole.” The movement in the opposite direction is dorsiflexion, wherein the dorsal part (top) of the foot is moved in a manner toward the tibia. It occurs at the ankle. The range of motion for plantar flexion is usually indicated in the literature as 30° to 40° but sometimes also 50°.

Thus, it has been shown that the apparatus of the invention possesses versatility and has a frame that can be adjusted and placed into various positions selected for various groups of a patient's muscles, depending on a specific condition or recovery stage.

With regard to the device of the invention, bending and stretching of the knee joint can be carried out with use of a knee-immobilization device. The patient's foot is placed on a foot pedal and drives the sliding carriage along the frame in the forward or reverse direction (i.e., in the leg-stretching or bending direction) by using motions of the foot around the ankle joint. The force of the foot is transmitted by means of the ratchet wheel to the drum, on which the one end of the cord is secured and onto which the cord is wound. The other end of the cord is guided by a pulley that is attached either to a stationary crossbar on the front part of the frame or on the carriage and is secured either to the rear crossbar or to the knee brace. Rotation is transmitted from the foot pedal to the ratchet wheel by means of a lever mechanism, and direction of the ratchet wheel rotation depends on the position of the ratchet mechanism. This mechanism has two ratchet pawls that can be switched in an alternating manner by a toggle mechanism between the first position, in which the first pawl engages one side of the ratchet wheel for rotation in the clockwise direction, and the second position in which the second pawl engages the ratchet wheel on the other side for rotation of the ratchet wheel in the counterclockwise direction. On the side facing the cord drum, the ratchet wheel has a first half clutch, while the second half clutch is formed on the facing side of the cord drum. The drum is slidingly supported on the ratchet wheel shaft and can be shifted in the axial direction of the ratchet shaft toward the ratchet to engage the half clutches and thus to transmit rotation of the ratchet to the cord drum.

Axial movement of the drum in the clutch engagement direction is carried out by means of a flexible steel cable, the control of which is in the patient's hands.

Thus, by switching positions of the pulley 100 between the front crossbar and the carriage and by securing the free end of the cord either to the rear crossbar or to the knee brace on the patient's leg, it is possible to move the sliding carriage with the foot secured on the foot pedal in the leg-stretching or leg-bending direction.

Knee bending and stretching exercises performed with the use of the apparatus of the invention provide the following effects:
1. Maintain muscle tone
2. Restore mobility in knee and hip joints
3. Improve blood circulation through tissue and leg muscles
4. Prevent formation of thrombuses in blood vessels of injured leg
5. Reduce edema in knee and hip joints
6. Prevent “freezing” of joints in post-operative period
7. Shorten time required for healing and restoration of normal functioning of knee and hip joints

The method of the invention for training knee and hip joints in the post-traumatic or post-operative period comprises use of the pivotal motions of the healthy foot of the injured or post-operative leg as a force for driving a carriage that supports the leg and causes, in the course of its motions, controlled stretching or bending of the injured or post-operative knee or hip. The method also comprises the step of manually controlling a clutch mechanism that connects the foot-driven device with the carriage movement mechanism. The method comprises the steps of performing the aforementioned motions of the leg-supporting carriage in various positions and conditions of the apparatus frame for causing operations of different groups of leg muscles.

Thus, it has been shown that the apparatus and method of the invention make it possible to use ankle pump motions of the healthy foot of a leg with an injured or post-operative knee and/or hip joint as a means for driving a carriage in which the injured leg is secured.

The carriage movement control means are held in the patient's arm, and this allows control of the leg motions. The apparatus is versatile in that it can be adjusted to various conditions of the patient, starting with exercise during the initial stages of healing when freedom of leg motion is restricted to exercise during the latest stages of recovery when the leg has a greater degree of freedom of motion.

Although the invention has been shown and described with reference to specific embodiments, it is understood that these embodiments should not be construed as limiting the areas of application of the invention and that any changes and modifications are possible provided that these changes and modifications do not depart from the scope of the attached patent claims. For example, the clutch engaging and disengaging mechanism can be embodied in a form different from that shown in the description and drawings. The frame can be foldable and can be made from rods of angular profile instead of the tubular rods shown in the drawings. Although it is not shown in the drawings, it is understood that the longitudinal bars 44a and 44b also have means for adjusting their length in order to match the height of the patient. The position of the rear crossbar on the frame is also adjustable. The seat can be easily connected and disconnected in various ways, and the pulley attachment mechanism can be embodied in dozens of various ways. During the latest stages of recovery, the apparatus can be used without knee braces.

1. An apparatus for training a patient's knee and hip joints in the post-traumatic or post-operative period comprising the following:
a frame with guides;
a carriage moveable in the guides of the frame;
a foot support pivotally installed on the carriage with possibility of performing pivotal ankle pump motions of the foot secured in the foot support;
a device for securing a foot on the foot support; and
a motion-conversion mechanism that connects the foot support with the carriage to convert ankle pump motions of the foot into linear motions of the carriage in the guides of the frame.

2. The apparatus of claim 1, further comprising a control mechanism for controlling operation of the motion-conversion mechanism.

3. The apparatus of claim 2, wherein the motion-conversion mechanism comprises a clutch-and-drum assembly that comprises a shaft installed on the carriage; a drum having a first end face, a second end face and being supported on the shaft for free rotation and axial motions along the shaft; a first half-clutch formed on the first end face of the drum; a sprocket installed on the shaft on the side of the first end face of the drum, the sprocket being supported for free rotation on the shaft; a second half-clutch formed on the sprocket for engagement with the first half-clutch; a first pawl pivotally supported on the foot support for engagement with the sprocket so that pivotal motions of the foot support cause rotation of the sprocket; and a cord wound on the drum.

4. The apparatus of claim 3, further comprising a resilient body that normally shifts the drum away from the sprocket to maintain the first half-clutch and the second half-clutch in the disengaged state.

5. The apparatus of claim 4, further comprising a second pawl and a third pawl for engagement with the sprocket, the second pawl and the third pawl being switchable between a first position in which the sprocket can rotate in the clockwise direction and a second position in which the sprocket can rotate in the counterclockwise direction, in both cases with drive from the first pawl, positions of which may be changed depending on the direction of the sprocket rotation.

6. The apparatus of claim 3, further comprising a cord-guiding-and-attachment device for attachment of said other end of the cord to the frame or to the carriage, the cord-guiding-and-attachment device comprising a pulley rotationally installed on an axle that is selectively attachable to a carriage or to a selected position on the frame.

7. The apparatus of claim 1, wherein the frame comprises longitudinal bars, a front crossbar that connects the longitudinal bars and is located on one side of the carriage; a rear crossbar that connects the longitudinal bars and is located on the other side of the carriage; and a seat for a patient located on the side of the rear crossbar.

8. The apparatus of claim 7, wherein the motion-conversion mechanism comprises a clutch-and-drum assembly that comprises a shaft installed on the carriage; a drum having a first end face, a second end face, and being supported on the shaft for free rotation and axial motions along the shaft; a first half-clutch formed on the first end face of the clutch; a sprocket installed on the shaft on the side of the first end face of the drum, the sprocket being supported for free rotation on the shaft; a second half-clutch formed on the sprocket for engagement with the first half-clutch; a first pawl pivotally supported on the foot support for engagement with the sprocket so that pivotal motions of the foot support cause rotation of the sprocket; and a cord wound on the drum and having one end secured on the drum and another end connected at least to the rear crossbar.

9. The apparatus of claim 8, further comprising a resilient body that normally shifts the drum away from the sprocket to maintain the first half-clutch and the second half-clutch in the disengaged state, and a control mechanism for controlling motions of the drum along the shaft toward the sprocket to bring the first half-clutch and the second half-clutch to the engaged state.

10. The apparatus of claim 9, further comprising a second pawl and a third pawl for engagement with the sprocket, the second pawl and a third pawl being switchable between a first position in which the sprocket can rotate in the clockwise direction and a second position in which the sprocket can rotate in the counterclockwise direction, in both cases with drive from the first pawl.

11. The apparatus of claim 8, further comprising: a knee brace applied to the leg for immobilization of the knee joint; and a cord-guiding-and-attachment device for attachment of said other end of the cord selectively either to the front crossbar, to the rear crossbar, or to the knee brace; said cord-guiding-and-attachment device comprising a pulley rotationally installed on an axle that is selectively attachable to the carriage or to the front crossbar.

12. The apparatus of claim 9, wherein the control mechanism for controlling motions of the drum along the shaft toward the sprocket comprises a drum-shifting mechanism installed on the carriage, a control lever, a flexible metal cable having one end attached to the control lever and the other end to the drum shifting mechanism, the drum shifting mechanism having a lever, one end of which is pivotally installed on the carriage and the other end is urged against the second end face of the drum so that when the control mechanism is activated, the lever pushes on the second end face of the drum against the force of the resilient member and shifts the drum to engagement of the first half-clutch with the second half-clutch, whereby rotation of the sprocket is transmitted to the drum, the cord is wound onto the drum, and the carriage with the foot secured in the foot support moves in the leg-stretching or leg-bending direction.

13. The apparatus of claim 12, wherein the drum-shifting mechanism further comprises a pull link that passes through the lever, the pull link having a head that rests on the outer surface of the lever and an elongated part that projects through the lever in the direction of the sprocket and parallel to the shaft; an L-shaped lever pivotally installed on the carriage, the L-shaped lever having a first arm connected to the elongated part of the pull link and a second arm connected to the end of the flexible metal cable so that motions of the flexible metal cable are converted into axial displacements of the clutch-and-drum assembly through the L-shaped lever, pull link, and lever against the force of the resilient member.

14. The apparatus of claim 4, further comprising at least one knee brace one end of which can be placed onto the patient's leg and the other end of which is pivotally secured to the foot support; the knee brace having a device for securing said other end of the cord.

15. The apparatus of claim 14, further comprising a second pawl and a third pawl for engagement with the sprocket, the second pawl and a third pawl being switchable between a first position in which the sprocket can rotate in the clockwise direction and a second position in which the sprocket can rotate in the counterclockwise direction, in both cases with drive from the first pawl.

16. The apparatus of claim 11, further comprising a control mechanism for controlling operation of the motion-conversion mechanism, wherein the control mechanism for controlling motions of the drum along the shaft toward the sprocket comprises a drum-shifting mechanism installed on the carriage, a control lever, a flexible metal cable having one end attached to the control lever and the other end to the drum-shifting mechanism, the drum-shifting mechanism having a lever, one end of which is pivotally installed on the carriage and the other end is urged against the second end face of the drum so that when the control mechanism is activated, the lever pushes on the second end face of the drum against the force of the resilient member and shifts the drum to engagement of the first half-clutch with the second half-clutch, whereby rotation of the sprocket is transmitted to the drum, the cord being wound onto the drum, and the carriage with the foot secured in the foot support movement of the leg in the leg-stretching or leg-bending direction.

17. A method for training knee and hip joints of a patient in the post-traumatic or post-operative period comprising:
providing an apparatus having a frame with guides, a carriage moveable in the guides of the frame, a foot support pivotally installed on the carriage with possibility of performing pivotal ankle pump motions of the foot secured in the foot support, a device for securing a foot on the foot support, and a motion-conversion mechanism that connects the foot support with the carriage for converting the angle pump motions of the foot into linear motions of the carriage in the guides of the frame; and using ankle pump motions of the healthy foot of the injured or post-operative leg as a force for driving a carriage that supports the leg.

18. The method of claim 18, further comprising the step of providing the apparatus with a control mechanism for controlling the motion-conversion mechanism and controlling the direction of motion of the carriage in the leg-stretching or leg-bending direction for performing leg stretching or leg bending.

19. The method of claim 18, further comprising the step of providing the apparatus with a knee brace, and connecting the knee brace to the carriage through a cord and pulling the knee brace down toward the frame for performing leg stretching.

20. The method of claim 19, further comprising the step of positioning the frame and the patient on the frame in various positions selected for training the knee and hip joints of the injured or post-operative leg of the patient by applying the load to selected groups of muscles.
